(19) United States
(12) Patent Application Publication
(10) Pub. No.: US 2014/0358080 A1
(43) Pub. Date: Dec. 4, 2014

(54) BUBBLE ENTRAPMENT DEVICE WITH VARIABLE VOLUME

(71) Applicant: CORK INSTITUTE OF TECHNOLOGY, Cork (IE)

(72) Inventor: Keith Bryan, Cork (IE)

(21) Appl. No.: 14/366,303

(22) PCT Filed: Dec. 14, 2012
(86) PCT No.: PCT/EP2012/075565

§ 371 Date: Jun. 18, 2014

(30) Foreign Application Priority Data
Dec. 20, 2011 (IE) 2011/0557

(51) Int. Cl. A61M 5/36; B01D 19/00

(52) U.S. Cl. CPC A61M 5/36; B01D 19/0036
USPC 604/122; 96/193

(57) ABSTRACT

A bubble entrapment device has a cylinder, a plunger, and a centre tube. Threads on the centre tube engage threads of the cylinder and the plunger respectively. The device has a chamber with a tapered end configuration provided by the fact that the cylinder has a funnel-shaped base and the plunger has a corresponding concave shape. Rotation of the tube handle while holding the cylinder causes the chamber to change in volume, but the chamber outlet remains in the centre of volume throughout. During use, the chamber will be retained at or near maximum volume, but near end of dose this may be reduced so that residual liquid is expelled. This is particularly advantageous for applications such as neo natal delivery systems, where dose volumes are small.

### INTRODUCTION

1. Field of the Invention

The invention relates to delivery of liquids to patients in medical equipment.

2. Prior Art Discussion

Our prior published PCT Specification No. WO2011/077420 describes a bubble entrapment device for an infusion line. It has a chamber outlet port located centrally in the chamber's volume. Because the flow is slowed down in the device with respect to the remainder of the flow line there is a tendency for gas such as air to escape as bubbles which rise to the highest level. Importantly, because the highest level in the chamber is above the level of the outlet, bubbles are unlikely to escape. U.S. Pat. No. 7,279,031 (D. W. Wright) describes an emboli elimination apparatus, in which the outlet is also located centrally in a chamber which has a wall with vents to allow gas to pass out. An outlet is located centrally to prevent bubble flow out through the outlet at any orientation.

An additional requirement for certain clinical situations is that very little residual liquid be retained in the infusion set. In some situations such as some neonate and paediatric treatments the residual volume should not exceed a fraction of one ml.

The invention is directed towards providing a bubble entrapment device which has at least the bubble entrapment aspects of the prior art, but also allows for full or near to full outflow of all liquid which enters.

### SUMMARY OF THE INVENTION

According to the invention, there is provided a liquid supply bubble entrapment device comprising:

a housing,

a plunger arranged to move in the housing to define a chamber,

a chamber inlet,

a chamber outlet, and

wherein the plunger and the housing are arranged to move mutually to vary volume of the chamber while keeping the outlet positioned within the chamber volume to prevent outflow of gas during use.

Because the outlet is within the chamber, gas which escapes as bubbles in the chamber will rise above the outlet level and so will not pass. Also where desired, such as near end of dose in medical applications the chamber volume may be reduced so that the contained liquid may be expelled, leaving little or no residual liquid after use.

In one embodiment, the plunger and the housing are adapted to maintain the outlet at substantially the chamber centre of volume for a range of chamber volumes.

In one embodiment, the inlet and the outlet are in a tube which extends through the chamber.

In one embodiment, the housing, the plunger, and the tube are movable relative to each other so that as the volume changes the outlet remains located within the chamber volume. In one embodiment, the housing, the plunger, and the tube are threadably engaged with pitches so that relative rotational movement causes movement of the outlet relative to the housing.

In one embodiment, the inlet is adjacent the outlet.

In one embodiment, the inlet and the outlet are approximately diametrically opposed.

In one embodiment, the tube has an inlet conduit extending from one end of the tube to an opening of the tube forming the inlet. Preferably, the tube has an outlet conduit extending from an opening of the tube forming the outlet to an end of the tube. In one embodiment, the tube is threadably engaged with the plunger.

In one embodiment, the tube is threadably engaged with the housing, and the thread pitches are arranged so that rotation of the tube relative to the plunger and the housing causes the plunger to move translationally in the housing to vary the chamber volume, one rotational direction causing volume increase and the opposite direction causing volume decrease.

In one embodiment, the housing and the plunger are configured with surfaces to provide a conical chamber shape at an end of the chamber towards which the plunger moves to reduce volume.

In one embodiment, the housing has threads which engage with threads of a nut, and the nut is connected to the plunger so that rotation of the nut causes translation of the plunger to vary the chamber volume. In one embodiment, the housing has external threads and the nut has internal threads.

In one embodiment, the nut includes finger grips for user rotation.

In one embodiment, the device includes graduation markings to indicate chamber volume change. Preferably, said graduation markings are on the housing.

In one embodiment, the housing includes features which engage with another part of the device to provide a haptic or audio operator feedback indicating degree of chamber volume change.

In one embodiment, the inlet and the outlet are in the housing and/or the plunger.

In one embodiment, the inlet and the outlet are in the housing, and the plunger has an internal conduit arranged to interconnect the inlet and the outlet for a closed position of the plunger. Preferably, the inlet is in the plunger and the outlet is in the housing. In one embodiment, the inlet and outlet are coaxial.

In one embodiment, the device is adapted to connect in a medical liquid supply system, such as an infusion system.

In another aspect, the invention provides a medical liquid supply system for delivery of liquid to the body, said system comprising supply tubing and a device as defined above in any embodiment connected to said supply tubing. In one embodiment, said system is an infusion system.

### DETAILED DESCRIPTION OF THE INVENTION

### Brief Description of the Drawings

The invention will be more clearly understood from the following description of some embodiments thereof, given by way of example only with reference to the accompanying drawings in which:

FIGS. 1 and 2 are perspective views of a bubble entrapment device of the invention at different positions;

FIGS. 3 to 5 are cross-sectional views for full, reduced, and zero chamber volume positions respectively;

FIGS. 6 to 12 are perspective views of parts of the device of FIGS. 1 to 5, namely a cylinder, a plunger, a nut, and a centre tube;

FIGS. 13 and 14 are perspective views of a further device of the invention, at full chamber volume and zero volume respectively;

FIGS. 15 to 17 are cross-sectional views of the device of FIGS. 13 and 14 at full, reduced, and zero chamber volume respectively;

FIGS. 18 to 23 are views of parts of the device of FIGS. 13 to 17, namely a plunger, a centre tube, and a cylinder;

FIGS. 24 and 25 are perspective views of a further bubble entrapment device of the invention at full and reduced volumes respectively;

FIGS. 26 to 29 are cross-sectional views of the device at various positions in use, in which FIG. 26 shows a closed position for initial air purging, FIG. 27 shows the device with full volume, FIG. 28 at reduced volume, and FIG. 29 at approaching zero volume;

FIGS. 30 and 31 are perspective views of a plunger of the device of FIGS. 24 to 29, and FIGS. 32 and 33 are perspective views of a cylinder of this device;

FIGS. 34 to 43 are views of a further device of the invention, in which FIGS. 34 to 36 are cross-sectional views for different chamber volumes, FIG. 37 is an overall perspective view, FIGS. 38 and 39 are perspective views of a cylinder; FIGS. 40 and 41 are perspective views of a plunger, and FIGS. 42 and 43 are perspective views of a nut of the device; and

FIGS. 44 to 53 are views of a still further device, in which FIG. 44 is an overall perspective view, FIGS. 45 to 47 are cross-sectional views for different chamber volumes (full, reduced, and zero), FIGS. 48 and 49 are perspective views of a cylinder of the device, FIGS. 50 and 51 are perspective views of a plunger of the device, and FIGS. 52 and 53 are perspective views of a nut of the device.

### DESCRIPTION OF THE EMBODIMENTS

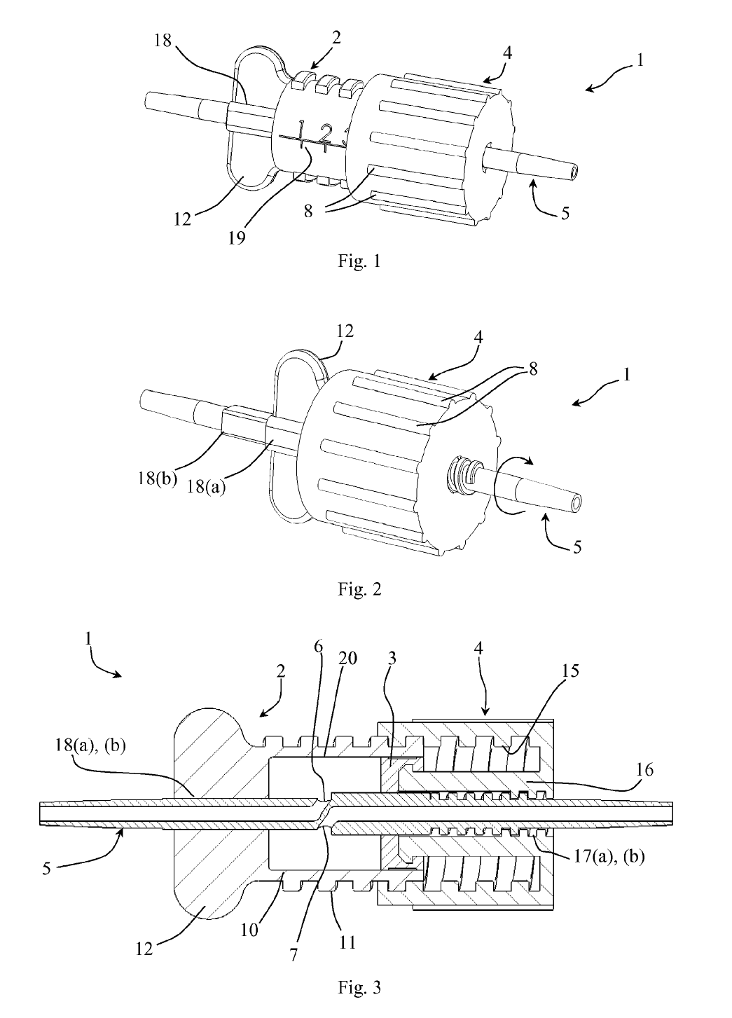

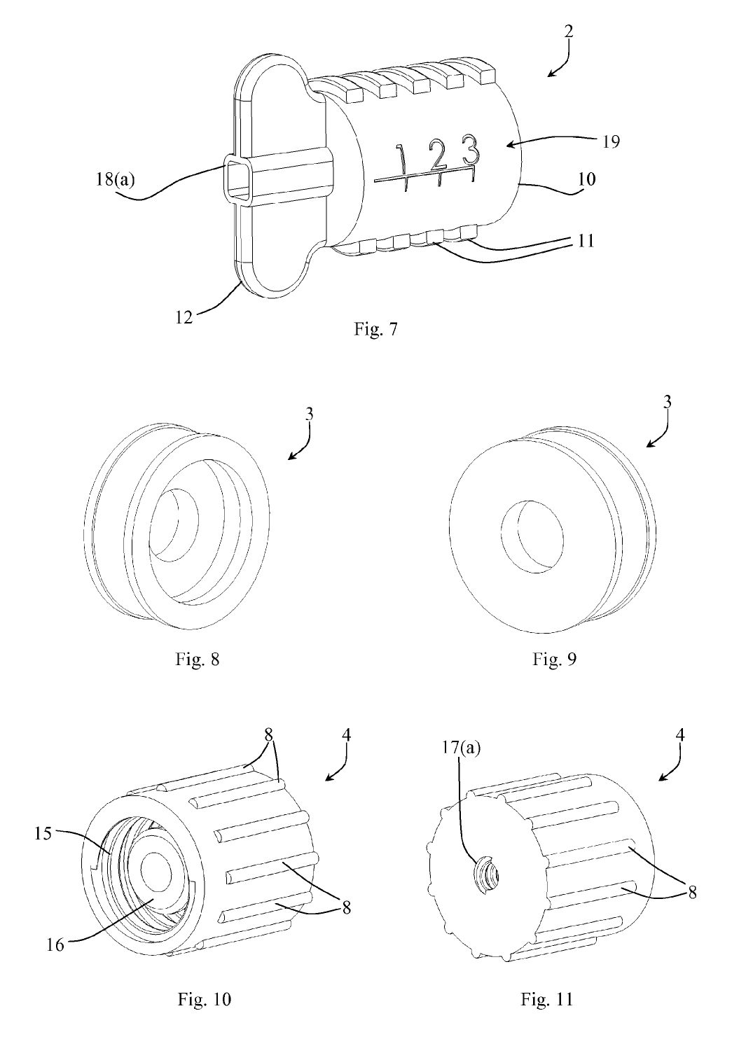

Referring to FIGS. 1 to 12 a bubble entrapment device 1 comprises a cylinder 2, a plunger 3, a thumbwheel nut 4, and a centre tube 5. The cylinder 2 has a main body 10 with external threads 11 and a handle 12. The cylinder 2 engages internal threads 15 of the nut 4, so that the nut 4 can be rotated by a user with the threads 11 and 15 engaging.

The nut 4 has a central sleeve 16 through which the centre tube 5 passes. The sleeve 16 and the centre tube are engaged by threads 17(a) on the nut 4 and 17(b) on the tube 5. The plunger 3 moves with the nut 4 and it seals off the sleeve 16 to form, with the internal surface of the cylinder 2, a chamber 20 of adjustable volume.

The centre tube 5 has separate inlet and outlet conduits in the form of central bores and terminating in an inlet 6 and an outlet 7 within the chamber 20. The roles of the inlet and the outlet may be reversed.

The cylinder handle 12 has a through hole 18(a) of substantially square cross-sectional shape. This receives a correspondingly-shaped part 18(b) of the tube 5 so that the tube 5 may slide through the handle 12 without rotating. The through-hole 18(a) and the tube part 18(b) are thus keyed together for axial movement only. In other embodiments different forms of key may be provided.

The cylinder 2 has graduation markings 19 for guiding the user on the extent of volume change of the chamber 20, in use.

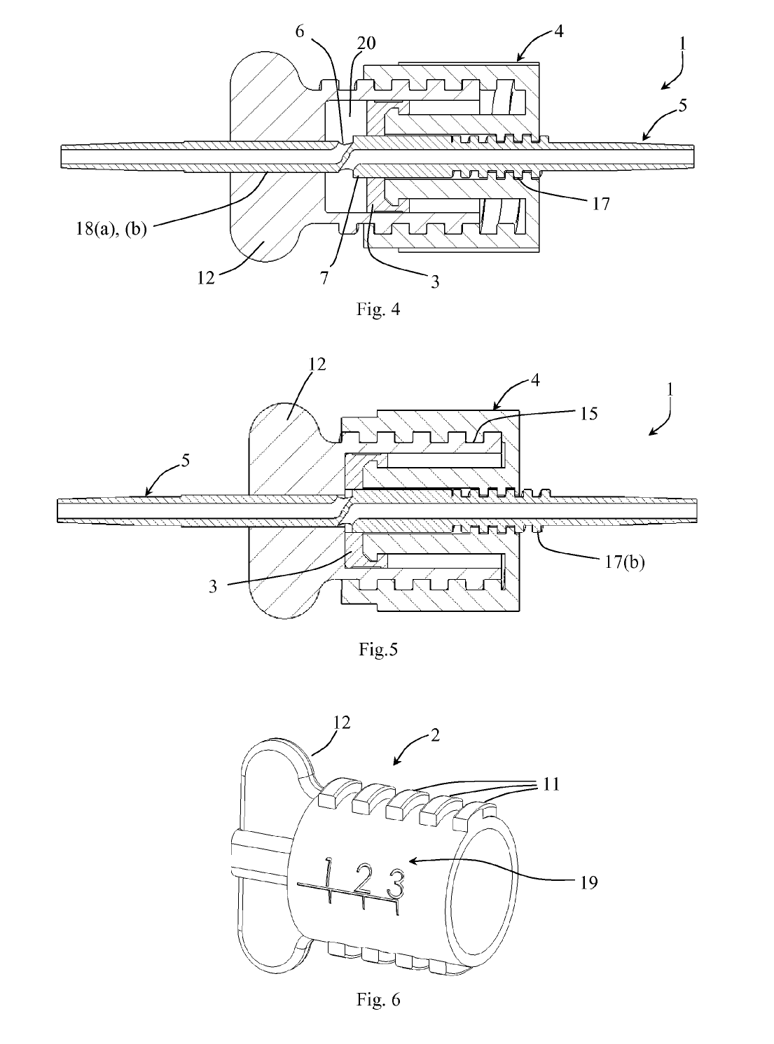

This chamber 20 has a volume which can be varied from full volume (FIG. 3) through an intermediate volume (FIG. 4) and to zero volume (FIG. 5) by gripping the handle 12 and rotating the thumbwheel nut 4. The threads 11/15 between the cylinder 2 and the nut 4 and the threads 17(a)/17(b) between the nut 4 and the tube 5 are of same orientations but of different pitches. The threads 11/15 have double the pitch of the threads 17(a)/17(b), so that for one rotation of the nut 4, the seal 3 travels X mm along the chamber 20 but the tube 5 only travels X/2 with respect to the nut 4. Therefore, as the nut 4 travels on the cylinder 2, the centre tube 5 travels through the centre of the nut 4 and the cylinder handle 12, thereby always keeping the inlet 6 and and the outlet 7 at the centre of the chamber. This is best seen in FIG. 4.

Therefore, the device 1 works as a bubble trap with the centre of volume principle at all chamber volume settings. It will also work in either flow direction because the fluid enters at the centre of volume and leaves at the centre of volume (but 180° apart on the tube 5).

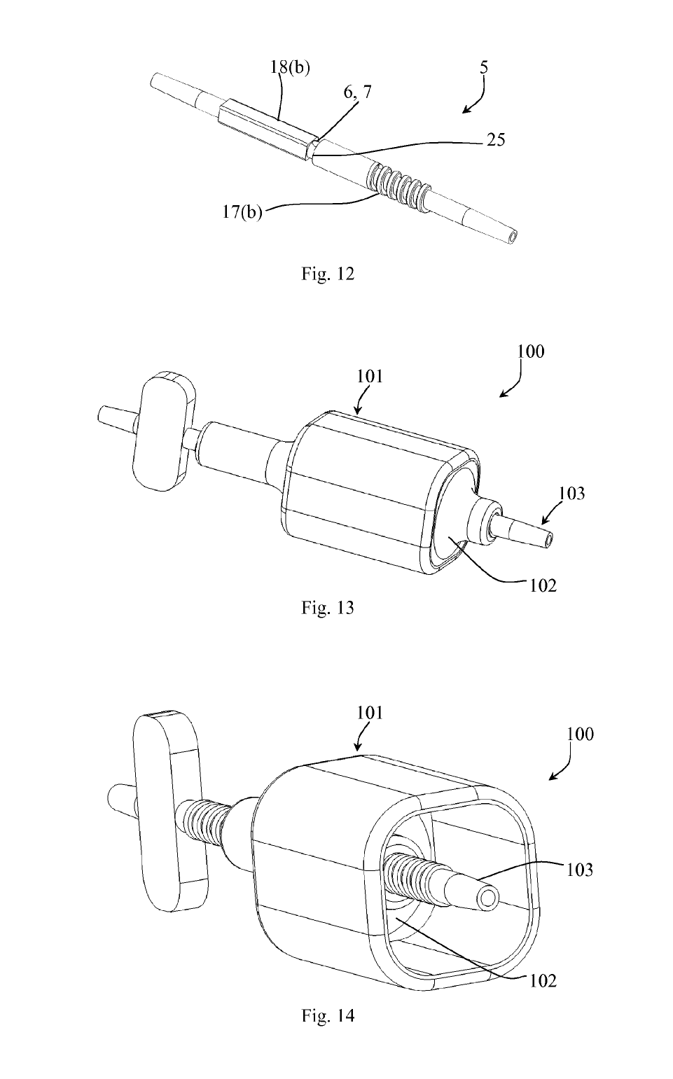

FIG. 3 shows the position of close to maximum chamber 20 volume. This is typically the starting volume for use, after purging the device of air with the chamber closed. Typically, the chamber volume will be left at the setting of FIG. 3 during almost all of a liquid delivery operation. FIG. 4 shows the positions when the chamber volume has reduced somewhat, and FIG. 5 shows the position for zero volume, and FIG. 12 shows the circumferential groove 25 which provides a straight-through path when fully closed. Typically, the chamber volume will be reduced through the setting of FIG. 4 and on to that of FIG. 5 when it is desired to expel all of the liquid at the end of a liquid delivery operation.

At the setting of FIG. 5 the inlet 6 and the outlet 7 are interconnected by the groove 25 around the tube 5, best shown in FIG. 12. Because the chamber 20 volume can reduce to zero, there will be little or no residual medication. Of course, if the device is used for administering a drug to a patient care must be taken to ensure that at the final stages of chamber volume reduction to zero bubbles do not pass out to the patient. The centre-of-volume bubble entrapment protection does not operate at close to zero volume. For medical applications such as infusion nurses are already well familiar with this aspect, using conventional devices such as syringes. Also, the graduations 19 are helpful for the operator to monitor the change in volume of the chamber during use.

Further, it is well known in this field that a typical peristaltic pump includes a sensor to detect bubbles and will emit a warning if bubbles are detected downstream of the device 1.

It is also envisaged that one or both of the inter-engaging pairs of threads may include features such as serrations to provide a haptic or audio feedback of the changing volume to the operator. For example, there may be one “click” per rotation or per 10 ml volume reduction.

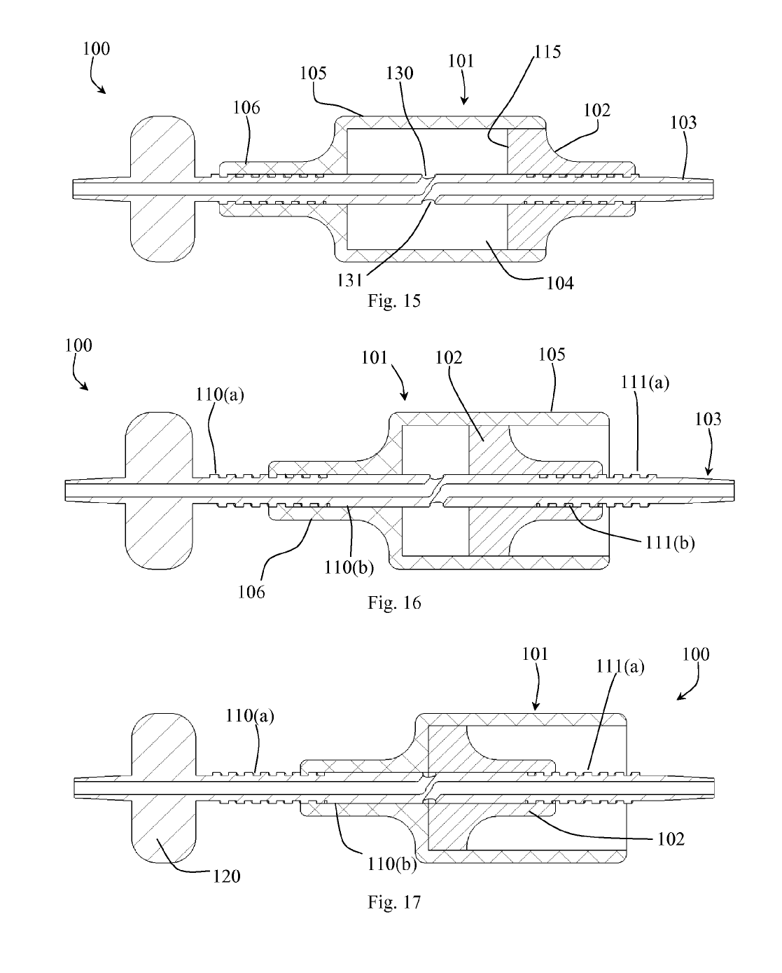

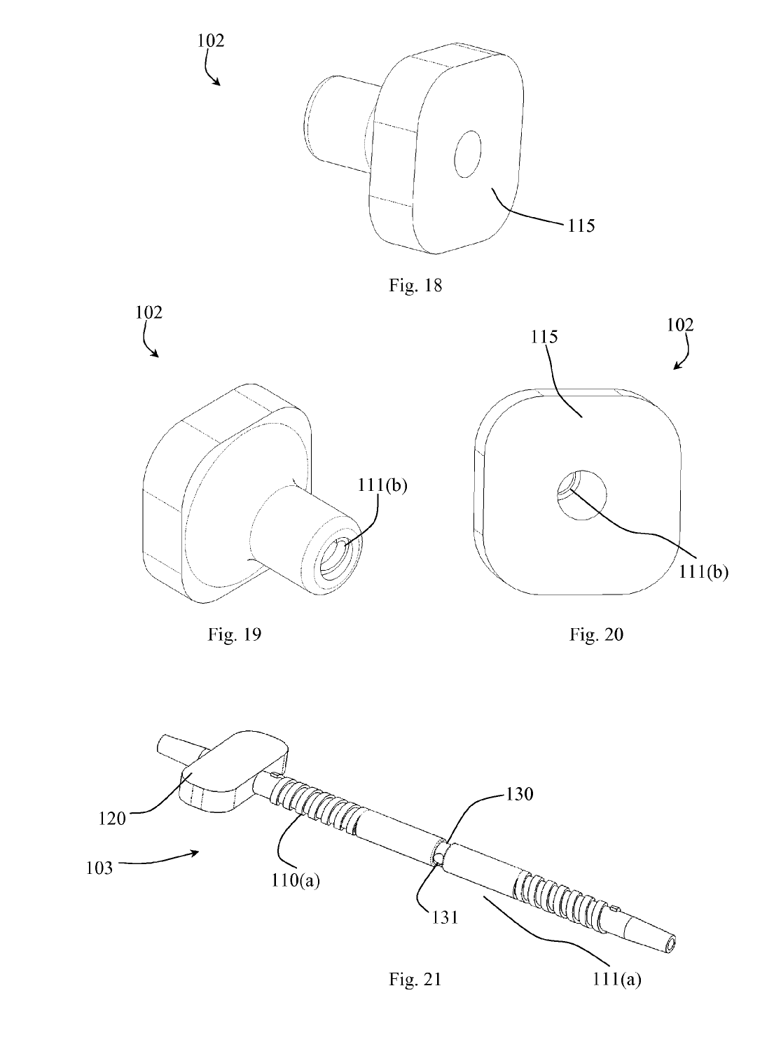

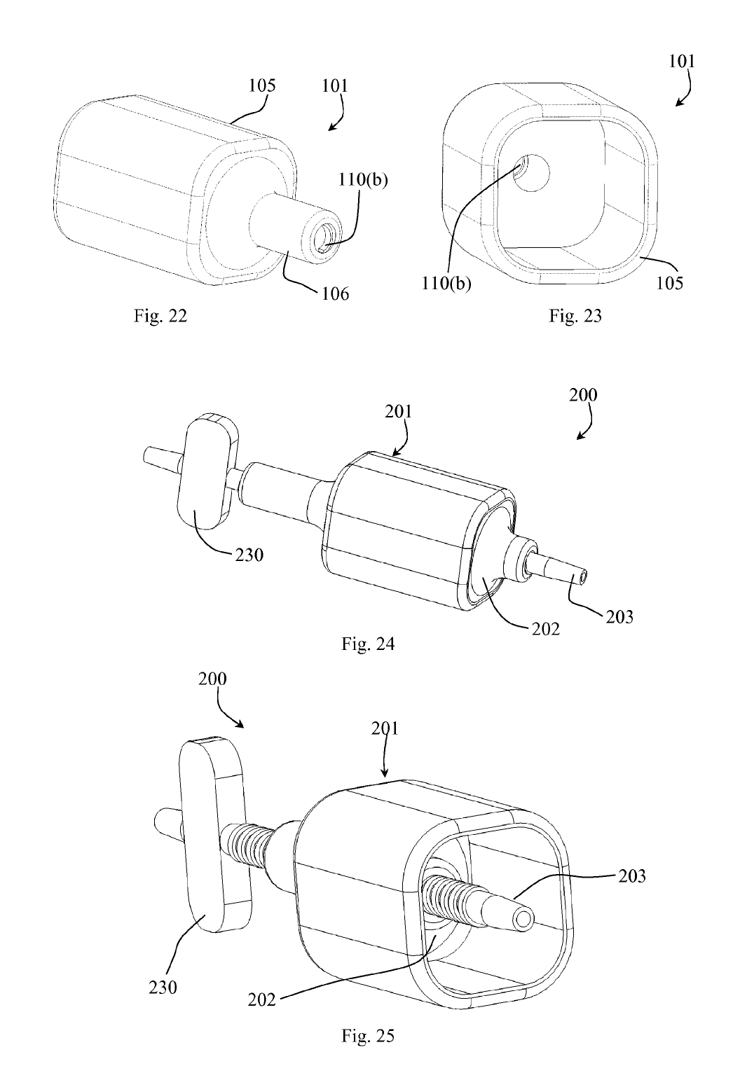

Referring to FIGS. 13 to 23 an alternative device 100 of the invention has a cylinder 101, a plunger 102, and a centre tube 103. A chamber 104 is defined by the inside surface of the cylinder 101 and an internal face 115 of the plunger 102. The cylinder 101 has a generally square cross-sectional shape so that the plunger is keyed to it to prevent mutual rotation. The tube 103 has a handle 120 at one end, rotation of which causes chamber volume variation.

The centre tube 103 has threads 110(a) engaging threads 110(b) of the cylinder 101, and threads 111(a) engaging threads 111(b) of the plunger 102. Because the cylinder 101 is shaped with a main body 105 of generally square cross-sectional shape with rounded corners, the plunger 102 will rotate with it and climb along the thread 111(a). Also, the cylinder 101 has a narrowed end forming a stem 106 which has the threads 110(b) engaging the threads 110(a) of the centre tube 103. The tube 103 terminates in the handle 120, for gripping of the device with the hand which is not gripping the cylinder 101 at the cylinder body 105.

The threaded portion of the centre tube 103 is outside the cylinder volume at full capacity, so the sealing portion of 101 and 102 are always on a smooth (non-threaded) portion of the tube 103.

The centre tube 103 has conduits along its length formed by central bores, terminating in a liquid inlet 130 and an outlet 131 within the chamber 104. Again, these roles are reversible.

The cylinder 101 and the plunger 102 are on left and right hand threads respectively, so they move in the opposite sense simultaneously with rotation of the tube 103 using the handle 120, while keeping the inlet and outlet 130 and 131 at the centre of volume of the chamber 104. Therefore, the bubble trap works with the centre of volume principle at all volume settings. This trap will work in either flow direction because the fluid enters at the centre of volume and also leaves at the centre of volume (but at 180° to the entry). While the centre of volume principle works at all volume settings it is more effective at larger volumes and in practice there is no need to reduce the volume until near the end of dose when it is desired to clear the device of liquid.

The device 100 has the benefit of not requiring production of an item such as the nut 4 of the device 1, which has multiple sets of threads, with different pitches and consists of only three main components.

Referring to FIGS. 24 to 33 a further device, 200, is illustrated. The device 200 is similar in principle to the device 100. It has a cylinder 201, a plunger 202, and a centre tube 203 with a handle 230. Again, threads 210(a) and 211(a) on the centre tube 203 engage threads 210(b) and 211(b) of the cylinder 201 and the plunger 202 respectively for operation on the same principle.

However, the chamber 225 formed by the cylinder 201 and the plunger 202 has a tapered end configuration. This is provided by the fact that the cylinder 201 has a funnel-shaped base 215 and the plunger 202 has a corresponding concave shape 216. This has the advantage of making it easier for the user to empty the liquid from the chamber 225 even if some air has accumulated in the chamber 225 during use. As illustrated, the tube 203 provides an inlet 226 which is an opening at the end of a tube conduit 226(a), and an outlet 227 which is an opening of the tube in the chamber 225 to a conduit 227(a). The outlet 227 (and less importantly, the inlet 226) remains centrally positioned in the chamber 225 throughout.

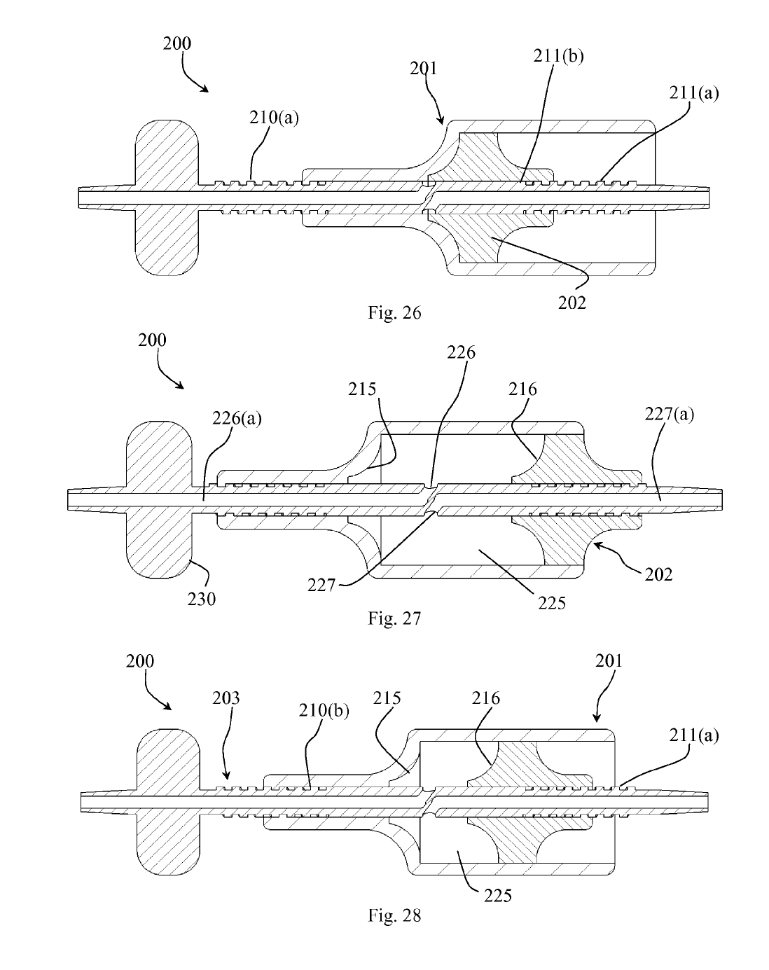

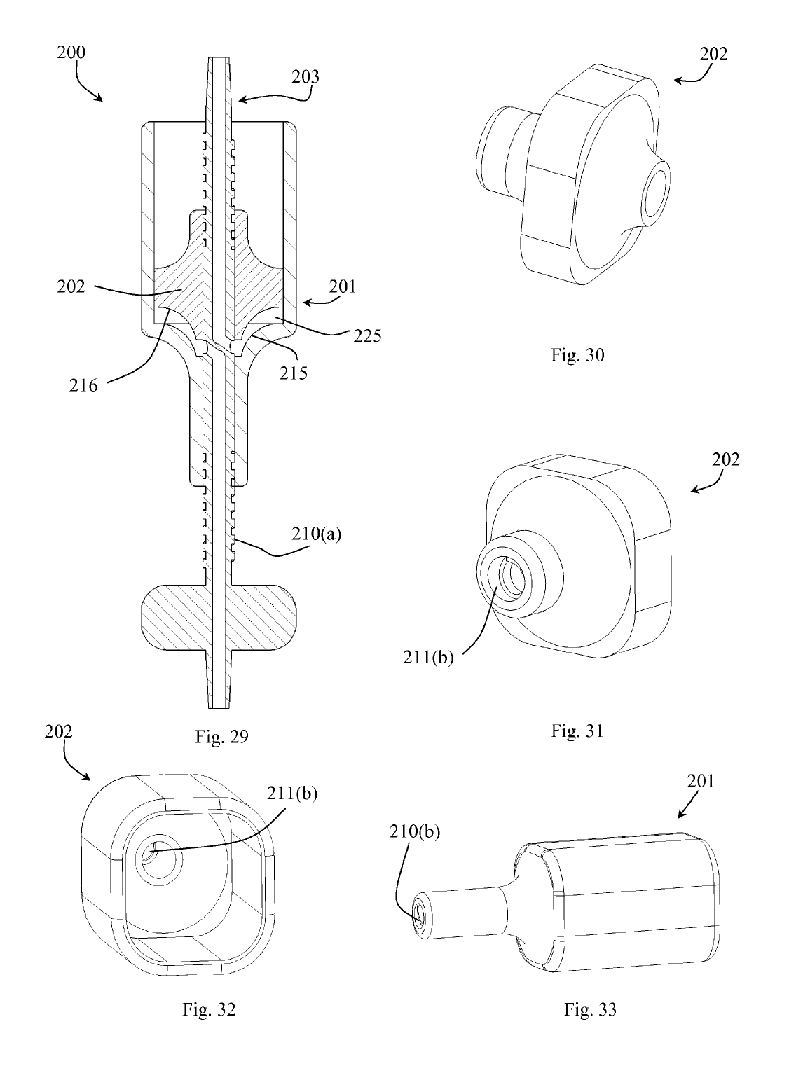

FIG. 26 shows the device 200 in the priming position, with straight-through flow to purge air before use. FIG. 27 shows the position at full volume during use. FIG. 28 shows the chamber at about half volume. FIG. 27 shows the trap in an almost-closed position and orientated so that any air will rise allowing the liquid to be expelled first. The device 200 is used in the same manner as the device 100, however, the tapered chamber end facilitates easier liquid expulsion. FIG. 29 shows how, if the device is orientated vertically, any bubbles will remain above the outlet as the liquid is expelled.

It will be appreciated that the invention achieves the benefits of being a very effective bubble trap, while also allowing expulsion of the liquid, leaving little or no residue.

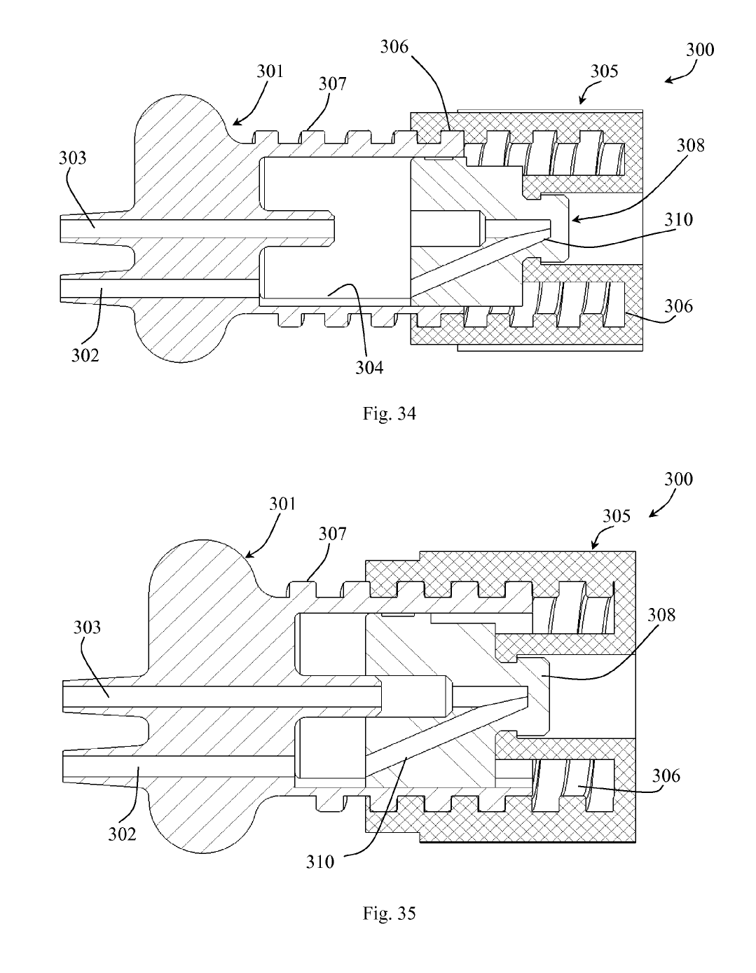

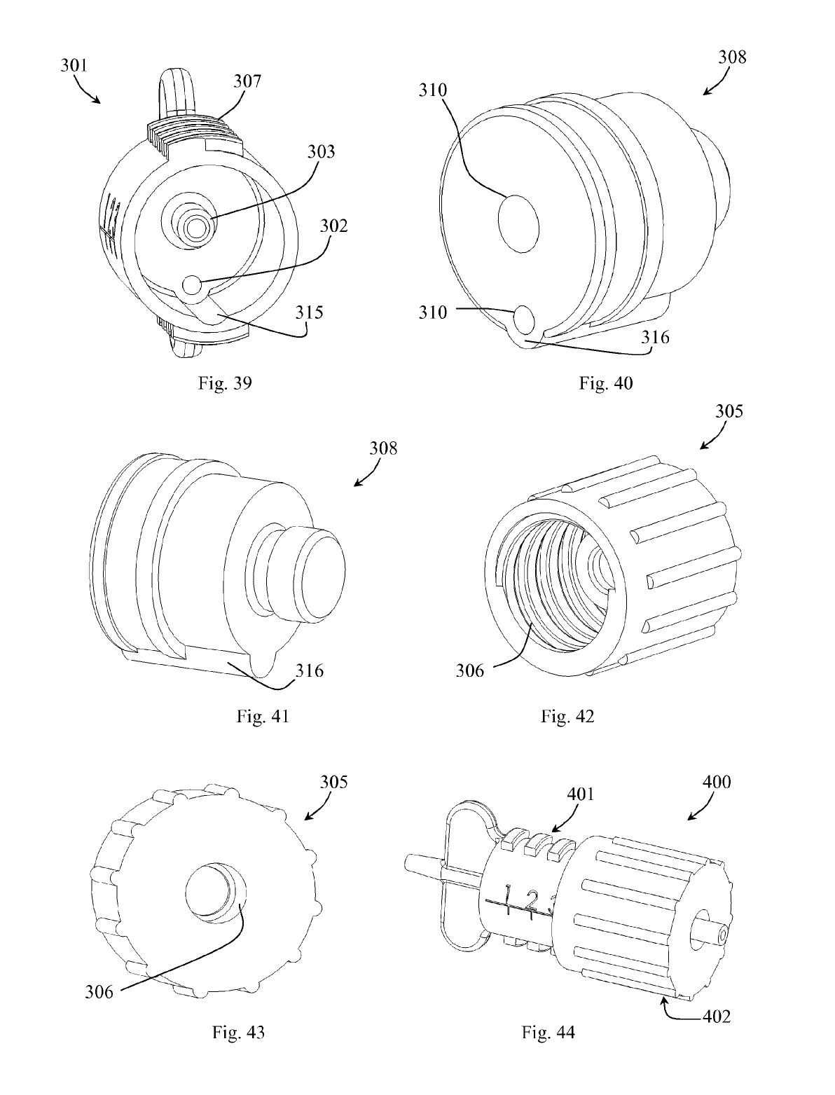

Referring to FIGS. 34 to 43 a device 300 has a cylinder 301 with a liquid inlet 302 and an outlet 303 to a chamber 304. A nut 305 has internal threads 306 engaging external threads 307 of the cylinder 301. A plunger 308 has an internal passageway 310 for liquid flow.

As illustrated, at the end of use, the volume can be reduced by turning the nut 305 relative to the handle 301. This is a controlled flow-rate because the cylinder 301 is graduated. At the position shown in FIG. 35, any bubbles accumulated are held in the chamber 304 while the liquid exits the chamber 304 at the bottom via the channel 310 in the plunger 308. The device 300 should be held in the orientation shown during emptying to ensure any bubbles will not exit.

The cylinder 301 has a groove 315 and the plunger 308 has a key 316 for keying of the plunger with the cylinder for mutual translational movement only.

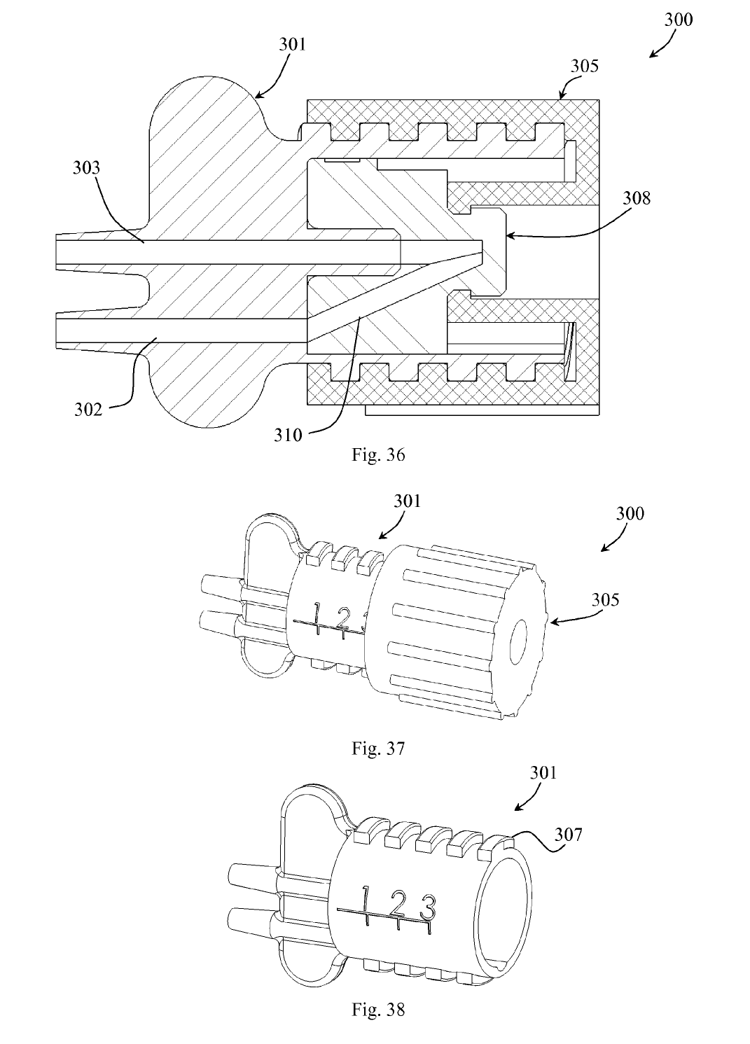

The zero volume position is shown in FIG. 36. As for the other embodiments this is used for initial purging, and when the chamber is then expanded it will fill with liquid and should not have any air bubbles If bubbles have accumulated during use the chamber 304 should not be completely reduced to zero, but if the unit is transparent, the user will see the bubbles. The device 300 does not maintain the chamber 304 outlet at the centre of volume throughout, however it does so for positions at or near the fully open position and this is the position used during use up to close to the end-of dose. The outlet 303 is within the volume so that bubbles will not exit for most of the range of chamber volumes, and as noted the device will typically have the chamber-open position as shown in FIG. 34 during use. It is only at the end of dose that the chamber volume is reduced.

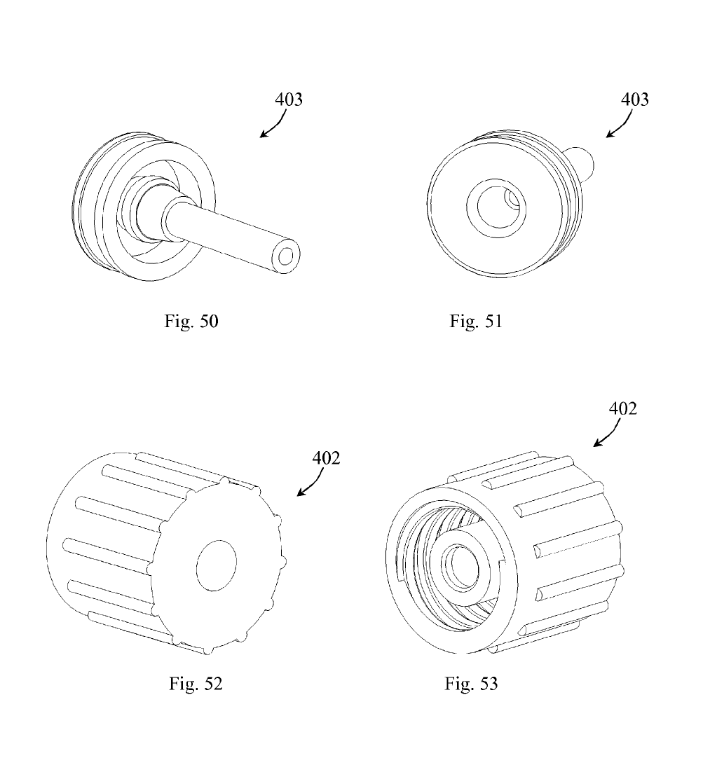

Referring to FIGS. 44 to 53 a further device, 400, comprises a cylindrical housing 401, a nut 402, and plunger 403. This device operates using the same principles as the device 300. The cylinder 401 and the plunger 403 form a chamber 410. In this case the plunger 403 has a liquid inlet 404 and there is an outlet 405 in the housing 401. The inlet 404 and the outlet 405 are aligned, however due to the manner of bubble movement it is very unlikely that bubbles would pass straight through. They would tend to track the chamber surface. Also, the in-use position is the maximum volume, at which the outlet 405 is in the centre of the chamber volume.

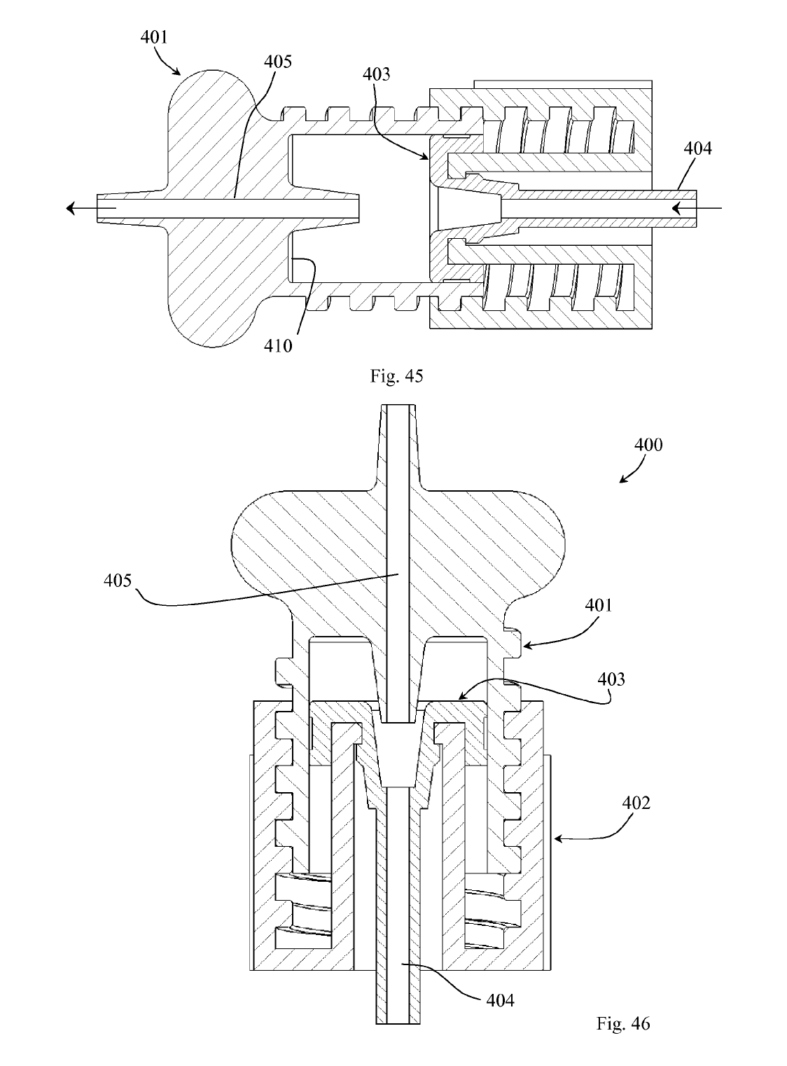

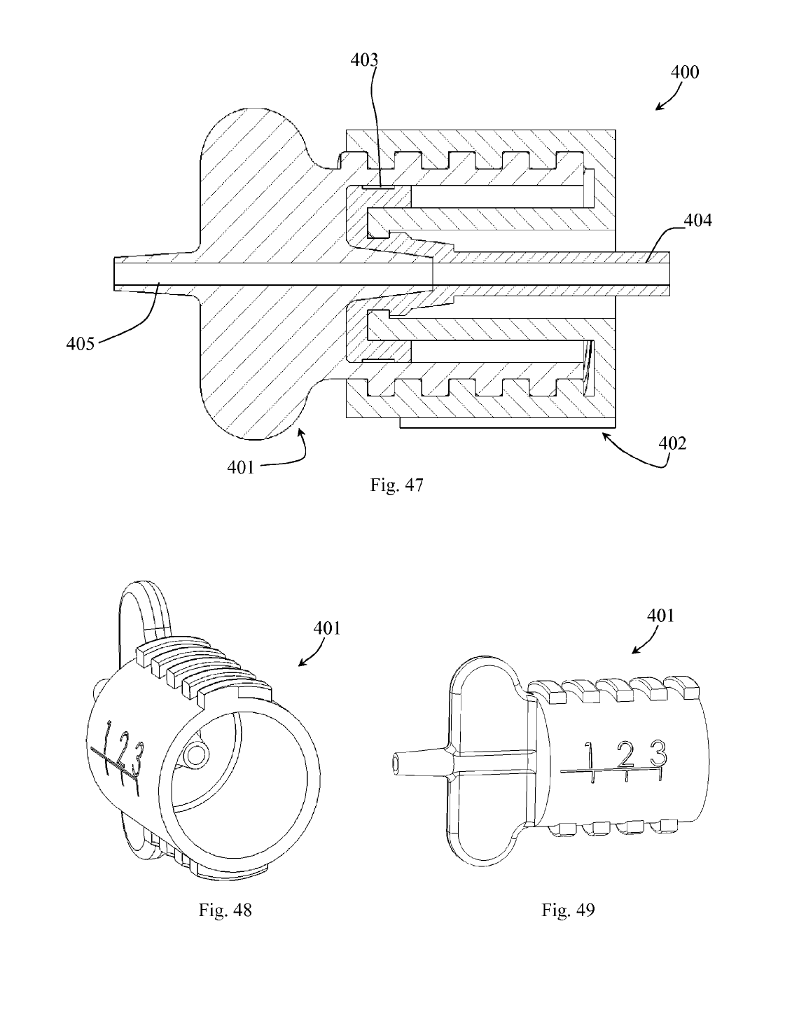

FIG. 45 shows the normal operating (fully open) position, in which flow is from right to left in this view. FIG. 46 shows the partially-closed position, which will typically be used only transitionally near the end of dose as residual liquid is expelled. In the upright position shown in FIG. 46 with the exit at the top, any bubbles accumulated will rise to the top of the chamber 410 and most or all of the liquid will be expelled before any gas will leave the volume. FIG. 47 shows the fully closed position. This would be the initial position for purging the system and the final position if no bubbles have accumulated. If any bubbles accumulated during use, the system would only be closed enough to expel the liquid and still hold the gas (as shown FIG. 46).

It will be appreciated that in the various embodiments the invention provides for bubble entrapment in a liquid supply such as a medical infusion line. It also allows expulsion of any remaining liquid. This is very advantageous, especially where the liquid volumes are small such as in neo natal applications, and hence it is desired to ensure that all of the dose is administered.

The invention is not limited to the embodiments described but may be varied in construction and detail. For example in the devices 100 and 200 which have a centre tube the inlet and outlet openings may not be 180° apart, depending on manufacturing criteria. It may be more feasible to have them say 90° apart. Indeed, for the embodiments having a centre tube, the inlet may not be in the centre tube, but rather through a housing wall into the chamber. It is the location of the outlet which is more important for gas entrapment. Also, the device may be used for other applications such as fuel lines.

1. A liquid supply bubble entrapment device comprising:
a housing,
a plunger arranged to move in the housing to define a chamber, a chamber inlet, a chamber outlet, and
wherein the plunger and the housing are arranged to move mutually to vary volume of the chamber while keeping the outlet positioned within the chamber volume to prevent outflow of gas during use.

2. A bubble entrapment device as claimed in claim 1, wherein the plunger and the housing are adapted to maintain the outlet at substantially the chamber centre of volume for a range of chamber volumes.

3. A bubble entrapment device as claimed in claim 1, wherein the inlet and the outlet are in a tube which extends through the chamber.

4. A bubble entrapment device as claimed in claim 3, wherein the housing, the plunger, and the tube are movable relative to each other so that as the volume changes the outlet remains located within the chamber volume.

5. A bubble entrapment device as claimed in claim 4, wherein the housing, the plunger, and the tube are threadably engaged with pitches so that relative rotational movement causes movement of the outlet relative to the housing.

6. A bubble entrapment device as claimed in claim 5, wherein the inlet is adjacent the outlet.

7. A bubble entrapment device as claimed in claim 6, wherein the inlet and the outlet are approximately diametrically opposed.

8. A bubble entrapment device as claimed in claim 3, wherein the tube has an inlet conduit extending from one end of the tube to an opening of the tube forming the inlet.

9. A bubble entrapment device as claimed in claim 3, wherein the tube has an outlet conduit extending from an opening of the tube forming the outlet to an end of the tube.

10. A bubble entrapment device as claimed in claim 3, wherein the tube is threadably engaged with the plunger.

11. A bubble entrapment device as claimed in claim 10, wherein the tube is threadably engaged with the housing, and the thread pitches are arranged so that rotation of the tube relative to the plunger and the housing causes the plunger to move translationally in the housing to vary the chamber volume, one rotational direction causing volume increase and the opposite direction causing volume decrease.

12. A bubble entrapment device as claimed in claim 3, wherein the housing and the plunger are configured with surfaces to provide a conical chamber shape at an end of the chamber towards which the plunger moves to reduce volume.

13. A bubble entrapment device as claimed in claim 1, wherein the housing has threads which engage with threads of a nut, and the nut is connected to the plunger so that rotation of the nut causes translation of the plunger to vary the chamber volume.

14. A bubble entrapment device as claimed in claim 13, wherein the housing has external threads and the nut has internal threads.

15. A bubble entrapment device as claimed in claim 13, wherein the nut includes finger grips for user rotation.

16. A bubble entrapment device as claimed in claim 1, wherein the device includes graduation markings to indicate chamber volume change.

17. A bubble entrapment device as claimed in claim 16, wherein said graduation markings are on the housing.

18. A bubble entrapment device as claimed in claim 1, wherein the housing includes features which engage with another part of the device to provide a haptic or audio operator feedback indicating degree of chamber volume change.

19. A bubble entrapment device as claimed in claim 1, wherein the inlet and the outlet are in the housing and/or the plunger.

20. A bubble entrapment device as claimed in claim 19, wherein the inlet and the outlet are in the housing, and the plunger has an internal conduit arranged to interconnect the inlet and the outlet for a closed position of the plunger.

21. A bubble entrapment device as claimed in claim 19, wherein the inlet is in the plunger and the outlet is in the housing.

22. A bubble entrapment device as claimed in claim 21, wherein the inlet and outlet are coaxial.

23. A bubble entrapment device as claimed in claim 1, wherein the device is adapted to connect in a medical liquid supply system.

24. A bubble entrapment device as claimed in claim 23, wherein said system is an infusion system.

25. A medical liquid supply system for delivery of liquid to the body, said system comprising supply tubing and a device of claim 1 connected to said supply tubing.

26. A medical liquid supply system as claimed in claim 25, wherein said system is an infusion system.
